(19) United States
(12) Patent Application Publication
(10) Pub. No.: US 2024/0277405 A1
(43) Pub. Date: Aug. 22, 2024

(54) ELECTRODE CATHETER

(71) Applicant: Japan Lifeline Co., Ltd., Tokyo (JP)

(72) Inventors: Kohei SAKAKI, Tokyo (JP); Makoto KATO, Tokyo (JP); Ryo ISHIDA, Tokyo (JP)

(21) Appl. No.: 18/537,148

(22) Filed: Dec. 12, 2023

(30) Foreign Application Priority Data
Feb. 16, 2023 (JP) 2023-022641

(51) Int. Cl. A61B 18/14

(52) U.S. Cl. CPC A61B 18/1492; A61B 2018/0016; A61B 2018/00267

(57) ABSTRACT

An electrode catheter includes a catheter shaft and an electrode assembly and a distal-side bundling component disposed at least partially distal to the catheter shaft. The electrode assembly includes a plurality of spline portions each having a proximal end portion bundled by the catheter shaft and a distal end portion bundled by the distal-side bundling component. The plurality of spline portions includes, when viewed from a distal side in an axial direction of the catheter shaft, a counterclockwise spline portion extending counterclockwise from a side of the distal end portion toward a side of the proximal end portion and a clockwise spline portion extending clockwise from the side of the distal end portion side toward the side of the proximal end portion.

### CROSS-REFERENCE TO RELATED APPLICATIONS

This application claims the benefit of priority to Japanese Patent Application Number 2023-022641 filed on Feb. 16, 2023. The entire contents of the above-identified application are hereby incorporated by reference.

### TECHNICAL FIELD

The present disclosure relates to an electrode catheter.

### BACKGROUND

JP 2016-507349 T discloses an electrode catheter including a catheter shaft and an electrode assembly and a distal-side bundling component that are at least partially provided distal to the catheter shaft. The electrode assembly includes a plurality of spline portions each having a proximal end portion bundled by the catheter shaft and a distal end portion bundled by a distal-side bundling component.

### SUMMARY

When the electrode assembly is brought into contact with a living tissue, each of the plurality of spline portions flexibly deforms. At this time, if the amount of deformation of each spline portion excessively increases, each spline portion is unintentionally partially separated from the living tissue, making it difficult to have a contact range of each spline portion with the living tissue. Thus, devisal to appropriately improve the shape retention of the electrode assembly has been awaited.

One object of the present disclosure is to provide an electrode catheter that can provide improved shape retention of the electrode assembly.

An electrode catheter of a first aspect of the present disclosure includes a catheter shaft and an electrode assembly and a distal-side bundling component disposed at least partially distal to the catheter shaft. The electrode assembly includes a plurality of spline portions each having a proximal end portion bundled by the catheter shaft and a distal end portion bundled by the distal-side bundling component. The plurality of spline portions includes, when viewed from a distal side in an axial direction of the catheter shaft, a counterclockwise spline portion extending counterclockwise from a side of the distal end portion toward a side of the proximal end portion and a clockwise spline portion extending clockwise from the side of the distal end portion toward the side of the proximal end portion.

The present disclosure can provide an electrode catheter that can provide improved shape retention of the electrode assembly.

### BRIEF DESCRIPTION OF DRAWINGS

FIG. 1 is an explanatory diagram regarding the usage scene of an electrode catheter according to an embodiment.

FIG. 2 is a side view schematically illustrating the electrode catheter of the embodiment.

FIG. 3 is a perspective view illustrating the electrode assembly of the embodiment together with surrounding structures.

FIG. 4 is a side view illustrating the electrode assembly of the embodiment together with surrounding structures.

FIG. 5 is a side cross-sectional view illustrating the surrounding structure of the electrode assembly of the embodiment.

FIG. 6 is a view of the electrode assembly of the embodiment viewed from the distal side in the axial direction.

FIG. 7 is a side view illustrating a clockwise spline portion and a counterclockwise spline portion of the embodiment together with surrounding structures.

FIG. 8 is a view of the clockwise spline portion and the counterclockwise spline portion of the embodiment, together with the surrounding structure, viewed from the distal side in the axial direction.

FIG. 9 is an explanatory diagram of an angular variation.

FIG. 10 is a side view of a grouping linear member of the embodiment.

FIG. 11 is a view seen from arrow A in FIG. 4.

FIG. 12 is a cross-sectional view taken along line B-B in FIG. 4.

FIG. 13 is a cross-sectional view taken along line C-C in FIG. 4.

### DESCRIPTION OF EMBODIMENTS

Hereinafter, embodiments for carrying out the present disclosure will be described. The same or equivalent constituent elements are denoted by the same reference signs, and redundant descriptions are omitted. In the drawings, for convenience of explanation, constituent elements are omitted, enlarged, or reduced, as appropriate. The drawings are to be viewed in accordance with the orientation of the reference signs.

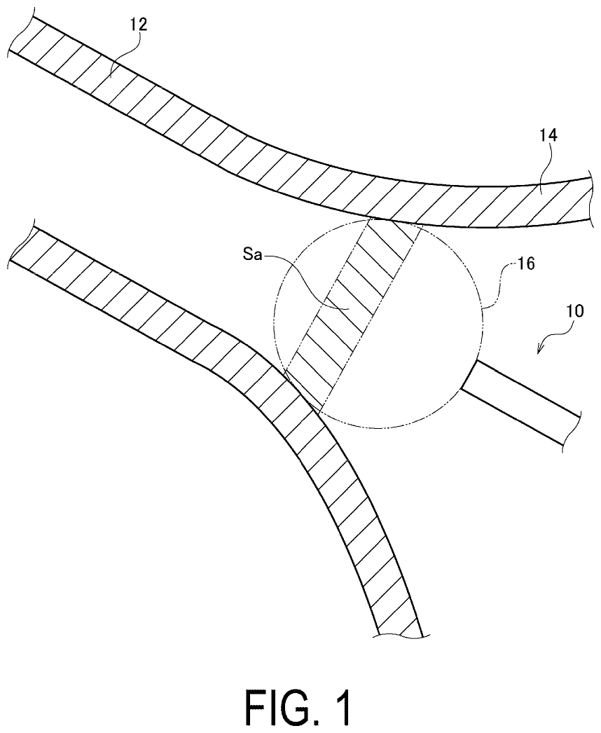

Description will be made with reference to FIG. 1. The usage scene of an electrode catheter 10 of the present embodiment will be explained first. The electrode catheter 10 is used for treatment of a living body. “Treatment” here refers to an act related to treatment or examination of a living body. The electrode catheter 10 of the present embodiment is used for treatment of atrial fibrillation by pulsed field ablation (PFA). Atrial fibrillation often occurs due to transmission of an abnormal electrical signal originating in the pulmonary vein 12 to the left atrium 14. This treatment is typically administered by annularly ablating the boundary between the pulmonary vein 12 and the left atrium 14 using an electrode assembly 16 of the electrode catheter 10. Here, the ablation range Sa by the electrode catheter 10 is hatched. This blocks transmission of an abnormal electrical signal from the pulmonary vein 12 to the left atrium 14. The energization method using the electrode assembly 16 may be a monopolar method in which energization is performed with a counter electrode placed outside the body, or a bipolar method in which energization is performed with another electrode placed inside the body.

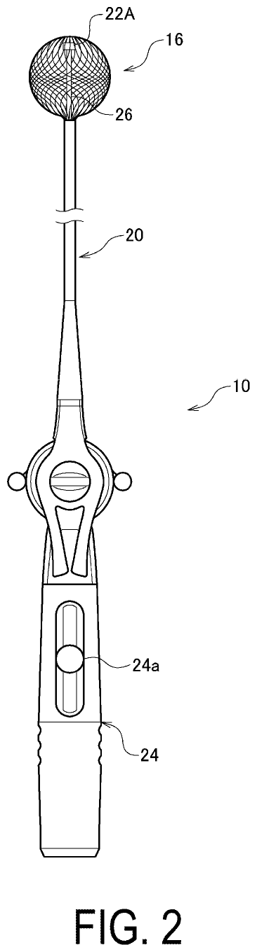

Description will be made with reference to FIG. 2. The electrode catheter 10 includes a catheter shaft 20, an electrode assembly 16 and a distal-side bundling component 22A that are at least partially provided distal to the catheter shaft 20, and a handle 24 attached to a proximal-side portion of the catheter shaft 20 and held by an operator. Additionally, the electrode catheter 10 optionally includes an elongated member 26 to which the distal-side bundling component 22A is secured.

Hereinafter, the positional relationship of each component will be described with reference to the state in which the catheter shaft 20 is not bent or deformed but extends linearly. The circumferential direction and radial direction of a circle concentric with the axis of the catheter shaft 20 are simply referred to as the “circumferential direction” and the “radial direction.” The term “distal-side” refers to the side farther from the hand of the operator holding the handle 24 in the axial direction of the catheter shaft 20, and the term “proximal-side” refers to the side opposite to the distal side in the axial direction.

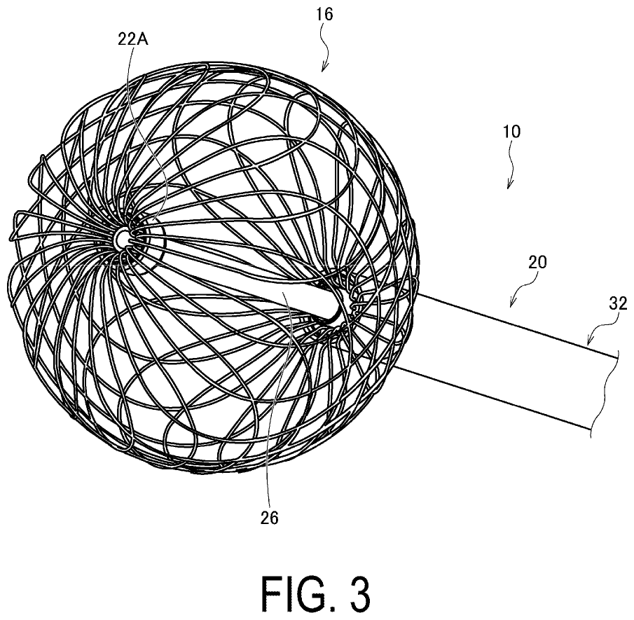

Description will be made with reference to FIGS. 3, 4, and 5. The catheter shaft 20 is inserted into the body at least at its distal end portion. The catheter shaft 20 is flexible enough to be bent and deformed. A main lumen 30 that continues into the handle 24 is formed in the catheter shaft 20. The catheter shaft 20 includes a shaft body 32 in which the main lumen 30 is formed, and the proximal-side bundling component 22B provided integrally with the shaft body 32. The shaft body 32 of the present embodiment is connected a plurality of shaft members 32a and 32b arranged in the axial direction by welding, adhesion, or the like. The proximal-side bundling component 22B is separate from the shaft body 32, and an example is illustrated in which it is arranged within the main lumen 30 and then fixed to the shaft body 32. Alternatively, the proximal-side bundling component 22B may be formed integrally with the shaft body 32. The catheter shaft 20 optionally includes a tube 36 that is inserted through the main lumen 30 of the shaft body 32.

The elongated member 26 extends in an elongated shape along the axial direction of the catheter shaft 20. The elongated member 26 is inserted through the main lumen 30 of the catheter shaft 20 and continues into the handle 24. The elongated member 26 in the present embodiment is formed of a shaft, but may also be a wire or the like. The elongated member 26 optionally includes a sub-lumen 26a formed inside the elongated member 26 along the axial direction. The sub-lumen 26a is used, for example, to insert and remove medical devices such as guide wires and other electrode catheters.

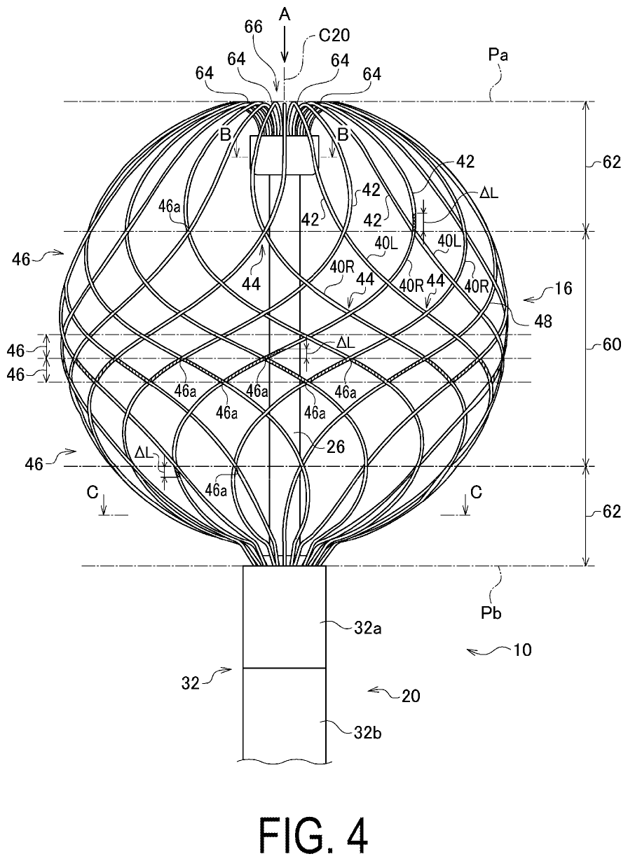

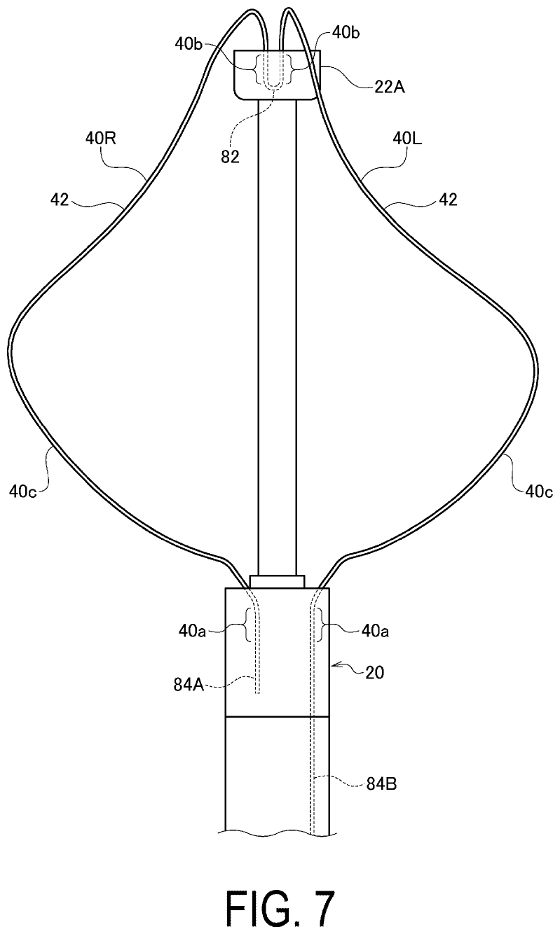

Description will be made with reference to FIGS. 4 to 8 (particularly FIGS. 4 and 7). The electrode assembly 16 includes a plurality of linear spline portions 40L and 40R having a linear shape. Although an example in which there are a total of thirty-two spline portions 40L and 40R is illustrated here, the number is not particularly limited. Spline portions 40L and 40R each include a proximal end portion 40a located at one end side in the longitudinal direction and a distal end portion 40b located at the other end side in the longitudinal direction. The longitudinal direction here refers to the direction along the axis of the spline portion 40L or 40R. The proximal end portions 40a of each of the plurality of spline portions 40L and 40R are bundled by the proximal-side bundling component 22B of the catheter shaft 20. The distal end portions 40b of each of the plurality of spline portions 40L and 40R are bundled by the distal-side bundling component 22A. The distal end portion 40b of the present embodiment is provided on the distal side from the proximal end portion 40a.

The spline portions 40L and 40R include a flexible portion 40c provided between the proximal end portion 40a and the distal end portion 40b in the longitudinal direction. The flexible portion 40c is flexibly bendable. The electrode assembly 16 can expand and contract in the radial direction by bending and deforming of the flexible portions 40c of each of the plurality of spline portions 40L and 40R. Expansion and contraction here refers to expansion toward the outer side in the radial direction and contraction toward the inner side in the radial direction. The electrode assembly 16 of the present embodiment can be expanded by the elongated member 26 pulling the distal-side bundling component 22A toward the proximal side. Alternatively, the electrode assembly 16 may be contracted by the elongated member 26 pushing the distal-side bundling component 22A toward the distal side. The elongated member 26 can move in conjunction with an operating member 24a (see FIG. 2) such as a slide knob provided on the handle 24, and the distal-side bundling component 22A can be pulled by the operator's operation of the operating member 24a.

The outer diameter of the electrode assembly 16 can be adjusted by expanding or contracting the electrode assembly 16 in the radial direction. The outer diameter of the electrode assembly 16 increases as the most distal position Pa (described later) of the spline portions 40L and 40R approaches the proximal side. In drawings such as FIG. 2, the maximum outer diameter within the adjustable range of the outer diameter of the electrode assembly 16 is illustrated. In the present embodiment, the outer diameter of the electrode assembly 16 is adjusted in accordance with the axial movement of the elongated member 26. The changing manner of the outer diameter of the electrode assembly 16 is an example, and may be changed in other manners.

An electrode portion 42 is provided in each of the plurality of spline portions 40L and 40R. The spline portions 40L and 40R of the present embodiment are provided with the electrode portions 42 on the entire outer circumferential surfaces thereof. To implement this, the spline portion 40L or 40R of the present embodiment is formed of a linear member composed of a conductive wire (electrode wire). The electrode portion 42 is used to apply current for treatment generated by an external power supply device to living tissues. In addition, the electrode portion 42 may also be used to take in a biological signal (for example, electrocardiographic potential) from living tissues.

The electrode catheter 10 described above has features related to (1) the electrode assembly 16 and (2) the end portions 40a and 40b of the respective spline portions 40L and 40R. First, the feature of (1) the electrode assembly 16 will be described.

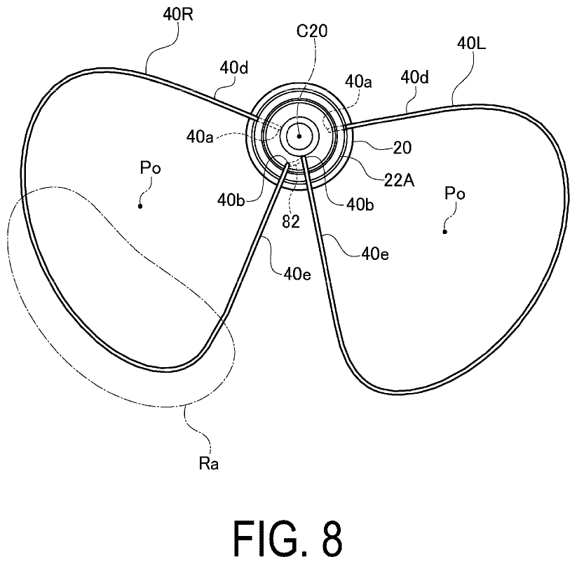

Description will be made with reference to FIGS. 7 and 8. The plurality of spline portions 40L and 40R includes a counterclockwise spline portion 40L extending counterclockwise from the distal end portion 40b side toward the proximal end portion 40a side and a clockwise spline portion 40R extending clockwise from the distal end portion 40b side toward the proximal end portion 40a side when viewed from the distal side in the axial direction. Each of the spline portions 40L and 40R is positioned radially offset from the axis C20 of the catheter shaft 20 and extends rotating clockwise or counterclockwise around a virtual offset point Po corresponding to each of the spline portions 40L and 40R. The spline portions 40L and 40R have a distal-side extension portion 40d extending radially outward from the distal end portion 40b and a proximal-side extension portion 40e extending radially outward from the proximal end portion 40a when viewed from the distal side in the axial direction. The spline portions 40L and 40R extend from the distal-side extension portion 40d toward the proximal-side extension portion 40e around the offset point Po.

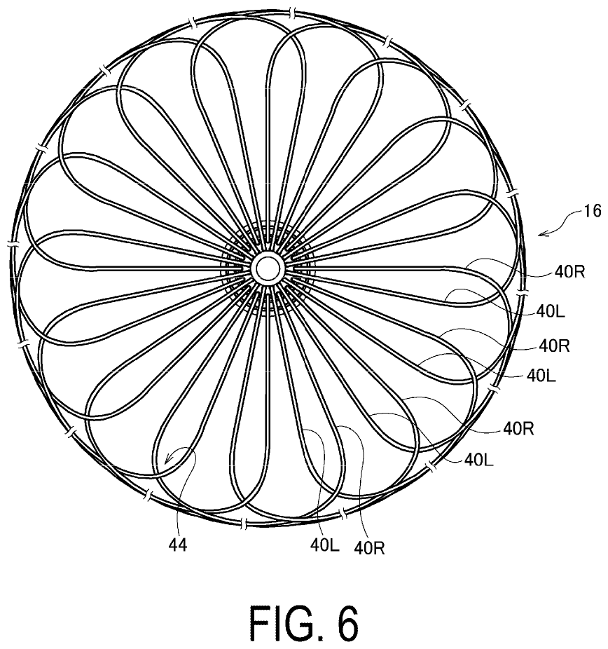

Description will be made with reference to FIGS. 4 and 6. FIGS. 4 and 6 illustrate only a part of the spline portions 40L and 40R visible on the front side of the paper and omit the other part thereof visible on the back side of the paper as appropriate. A plurality of the counterclockwise spline portions 40L is arranged at intervals in the circumferential direction. A plurality of the clockwise spline portions 40R is arranged at intervals in the circumferential direction. Although an example in which there are a total of sixteen clockwise spline portions 40R and a total of sixteen counterclockwise spline portions 40L is illustrated here, the number is not particularly limited.

The counterclockwise spline portion 40L and the clockwise spline portion 40R are provided in this manner, allowing the counterclockwise spline portion 40L and the clockwise spline portion 40R to be provided intersecting each other in a contactable manner. This means that a portion of the counterclockwise spline portion 40L and a portion of the clockwise spline portion 40R are provided intersecting and overlapping in the radial direction. FIG. 4 illustrates an example of such intersections 44 of the spline portion 40L and 40R. To implement this, each of the spline portions 40L and 40R of the present embodiment is in contact at the intersection 44. In addition, the respective spline portions 40L and 40R may be provided with a slight spacing at their mutual intersections 44 so that when an external force directed radially inward is applied to the outer spline portions, the outer spline portion can come into contact the inner spline portion with deformation of the outer spline portion.

The electrode assembly 16 has a mesh shape with the plurality of the clockwise spline portions 40R and the plurality of the counterclockwise spline portions 40L. As described above, this is achieved by providing the plurality of the clockwise spline portions 40R and the plurality of the counterclockwise spline portions 40L in a contactable manner. The mesh shape formed by the electrode assembly 16 is a shape in which a plurality of rows of zigzag patterns 46 extending in the circumferential direction is arranged in the axial direction. FIG. 4 illustrates a range of two rows of zigzag patterns 46 and hatches one row of zigzag patterns 46. The zigzag pattern 46 extends axially in zigzags in the circumferential direction. In the zigzag pattern 46, a bent portion 46a axially folded back in the circumferential direction is formed at the intersection 44 of the spline portions 40L and 40R. A plurality of the bent portions 46a is formed at intervals in the circumferential direction. The mesh shape formed by the electrode assembly 16 forms a plurality of rhombic meshes arranged in the axial direction and in the circumferential direction.

Either the counterclockwise spline portion 40L or the clockwise spline portion 40R may be on the outer side in the radial direction at the mesh-shaped intersection 44 formed by the electrode assembly 16. In the present embodiment, in the longitudinal direction of one counterclockwise spline portion 40L, an intersection 44 (hereinafter referred to as a first intersection 44) at which the counterclockwise spline portion 40L is on the outer side in the radial direction, and an intersection 44 (hereinafter referred to as a second intersection 44) at which the clockwise spline portion 40R is on the outer side in the radial direction are provided alternately will be described. In addition, the first intersections 44 and the second intersections 44 may be randomly provided in the longitudinal direction of one counterclockwise spline portion 40L. In relation to the shape retention of the electrode assembly 16 (to be described later), it is preferable that at least one first intersection 44 and at least one second intersection 44 are provided in the longitudinal direction of one counterclockwise spline portion 40L.

The electrode assembly 16 can form a continuous conductive path 48 that is continuous with the electrode portion 42 of the clockwise spline portion 40R and the electrode portion 42 of the counterclockwise spline portion 40L. As described above, this is achieved by bringing the counterclockwise spline portion 40L and the clockwise spline portion 40R into contact with each other, thereby energizing the electrode portions 42 of the respective spline portions 40L and 40R. The continuous conductive path 48 becomes a place through which an electric current can pass, and is conductively connected to the living tissue by bringing it into contact with the living tissue.

The electrode assembly 16 forms the afore-mentioned mesh shape in the continuous conductive path 48. The continuous conductive path 48 is continuous along the entire circumference around the axis C20 of the catheter shaft 20. This is achieved by having at least one row of zigzag patterns 46 included in the continuous conductive paths 48 continuous along the entire circumference. In the present embodiment, the mesh shape formed by the continuous conductive path 48 is continuous along the entire circumference around the axis C20 of the catheter shaft 20.

The electrode assembly 16 described above has the shape of a rotating body as a whole, centered on the axis C20 of the catheter shaft 20 due to the plurality of spline portions 40L and 40R. The rotating body here refers to a three-dimensional shape obtained by rotating a planar curve about the axis C20. The term “shape” used herein includes not only a shape that geometrically strictly corresponds to the shape indicated by the term immediately preceding it (a rotating body, a sphere, or the like), but also a shape that resembles that shape as a whole. The electrode assembly 16 has a spherical shape as such a rotating body. The term “sphere” as used herein includes spheroids such as oblate spheroids and prolate spheroids. It can be said that the electrode assembly 16, as a whole, has a spherical shape in which the outer diameter gradually increases and then gradually decreases from the distal side to the proximal side in the axial direction.

The effect of the electrode catheter 10 regarding the feature (1) will be described. The electrode assembly 16 includes the counterclockwise spline portion 40L and the clockwise spline portion 40R. This can provide the counterclockwise spline portion 40L and the clockwise spline portion 40R in a contactable manner, thereby improving the shape retention of the electrode assembly 16.

For example, consider a case where an external force directed inward in the radial direction is applied to the outer spline portions 40L and 40R around the contact point (intersection 44) between the counterclockwise spline portion 40L and the clockwise spline portion 40R. In this case, the external force can be transmitted from the outer spline portions 40L and 40R to the inner spline portions 40L and 40R through the contact points between the counterclockwise spline portion 40L and the clockwise spline portion 40R. Furthermore, by both the counterclockwise spline portion 40L and the clockwise spline portion 40R resisting against the external forces, the shape retention of the electrode assembly 16 can be appropriately enhanced. In particular, in relation to this effect, it is preferable that the electrode assembly 16 has a mesh shape with the plurality of the clockwise spline portions 40R and the plurality of the counterclockwise spline portions 40L. In this way, appropriately increasing the shape retention of the electrode assembly 16 can suppress, when the electrode assembly 16 is brought into contact with the living tissue, an excessive increase in the amount of deformation and makes it easier to allow the contact range of each of the spline portions 40L and 40R with the living tissue. In particular, this is advantageous in that it becomes easier to secure the conduction range of the electrode portions 42 provided in each of the spline portions 40L and 40R of the electrode assembly 16 to living tissues.

The electrode assembly 16 of the present embodiment can form the continuous conductive path 48 that is continuous with the electrode portion 42 of the clockwise spline portion 40R and the electrode portion 42 of the counterclockwise spline portion 40L. Thus, bringing the continuous conductive path 48 of each of the spline portions 40L and 40R into contact with the living tissue allows the conduction point (contact point of the electrode portion 42) of each of the spline portions 40L and 40R with the living tissue to be made continuous without any interval. As a result, it is possible to easily expand the circumferential range of continuous conduction points of the electrode assembly 16 with respect to the living tissue. Particularly in the case of ablation using the electrode catheter 10 as in the present embodiment, the more the circumferential range of continuous conduction points of the electrode assembly 16 with respect to the living tissue is expanded, the more advantageously the living tissue is annularly cauterized. In relation to this effect, it is preferable that the continuous conductive path 48 of the electrode assembly 16 is continuous along the entire circumference.

The electrode assembly 16 has a mesh shape in the continuous conductive path 48. Therefore, simultaneously bringing a plurality of rows of zigzag patterns 46 included in the mesh shape formed by the continuous conductive path 48 into contact with the living tissue allows the circumferential range of continuous conduction points relative to the living tissue to be stably expanded compared to the case of only one row of zigzag patterns 46.

When the mesh-shaped portion of the electrode assembly 16 is configured from an integrally molded product, the integrally molded product can be obtained by cutting the mesh-shaped portion of the workpiece by laser processing or other cutting processing on the workpiece. In this case, the degree of difficulty in manufacturing increases significantly due to the cutting processing. In this regard, in the electrode assembly 16 of the present embodiment, the mesh-shaped portion of the electrode assembly 16 can be formed of a plurality of spline portions 40L and 40R. Therefore, a mesh-shaped portion can be obtained by an assembly operation using a plurality of linear members forming the plurality of spline portions 40L and 40R. Therefore, in order to obtain the mesh-shaped portion, there is no need for cutting, which increases the difficulty in manufacturing, and thus, the difficulty in manufacturing can be reduced.

Other features related to (1) above will be explained. The electrode assembly 16 includes an intermediate region 60 forming a mesh shape and a pair of end regions 62 provided on respective both sides of the intermediate region 60 in the axial direction. The intermediate region 60 is provided in an axial range from a bent portion 46a of the zigzag pattern 46 which is on the most distal side in the axial direction to the bent portion 46a of the zigzag pattern 46 which is on the most proximal side. The distal-side end region 62 is provided in an axial range from the most distal position Pa on the most distal side in the axial direction, of the flexible portions 40c of the spline portions 40L and 40R to the bent portion 46a of the zigzag pattern 46. The proximal-side end region 62 is provided in an axial range from the bent portion 46a of the zigzag pattern 46 located on the most proximal side to the most proximal position Pb that is located on the most proximal side in the axial direction of the flexible portions 40c of the spline portions 40L and 40R and is exposed to the outside viewed along the radial direction. The most proximal position Pb will be outside the main lumen 30 when part of the spline portions 40L and 40R is inserted into the main lumen 30 of the catheter shaft 20.

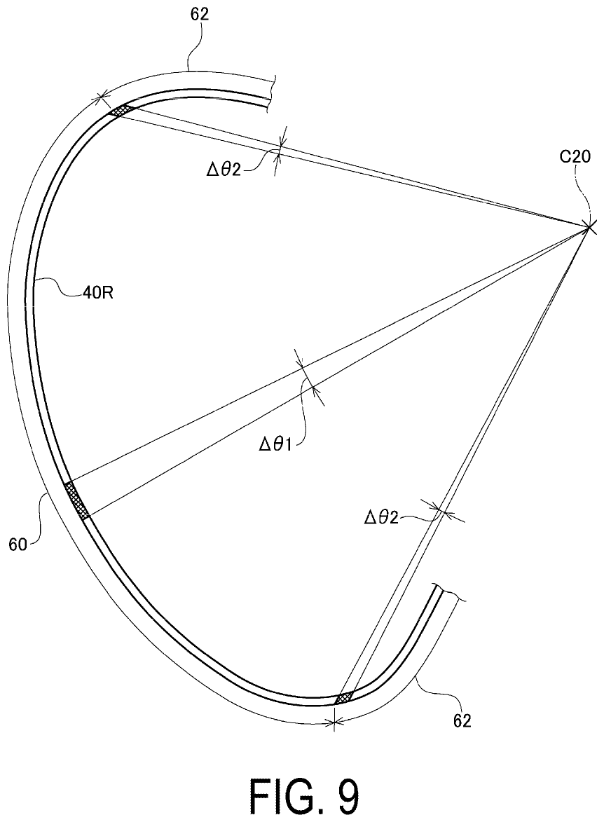

Description will be made with reference to FIGS. 4 and 9. FIG. 9 is an enlarged view of the range Ra in FIG. 8. Angular variations Δθ1 and Δθ2 per unit axial length ΔL of the spline portions 40L and 40R are defined. Δθ1 is the angular variation in the intermediate region 60, and Δθ2 is the angular variation in the end region 62. FIG. 9 adds double hatching to each of the unit axial lengths ΔL of the intermediate region 60 and end region 62 illustrated in FIG. 4. The angular variations Δθ1 and Δθ2 refer to the amount of change in angle around the axis C20 of the catheter shaft 20 of the virtual point on the spline portion 40L or 40R when the virtual point on the spline portion 40L or 40R advances in the axial direction along the spline portion 40L or 40R by the unit axial length ΔL. This unit axial length ΔL is a length that is sufficiently shorter (for example, one-hundredth of the total axial length) than the total axial length from the most distal position Pa to the most proximal position Pb of the spline portion 40L or 40R. FIG. 4 illustrates the unit axial length ΔL is illustrated in an exaggerated manner for convenience of explanation. When assuming these angular variations Δθ1 and Δθ2, only the angular variation when proceeding in the axial direction along the spline portion 40L or 40R in the range from the most distal position Pa to the most proximal position Pb of the spline portion 40L or 40R is considered. This means that the portion of the flexible portion 40c of the spline portion 40L or 40R on the distal end portion 40b side from the most distal position Pa is not considered. The larger the angular variations Δθ1 and Δθ2, the smaller the inclination angle of the unit axial length ΔL on the spline portion 40L or 40R with respect to the axially orthogonal plane.

The angular variation Δθ1 in at least part of the intermediate region 60 is larger than the maximum angular variation Δθ2(max) in the end region 62. The maximum angular variation Δθ2(max) refers to the maximum angular variation Δθ2 at each unit axial length ΔL of the end region 62. In the present embodiment, the location where the angular variation Δθ2(max) is maximized is provided at the end portion of the end region 62 near the boundary with the intermediate region 60. This angular variation Δθ1 is larger than the maximum angular variation Δθ2(max) in each of the pair of end regions 62. In the present embodiment, the angular variation Δθ1 in the entire region of the intermediate region 60 is larger than the maximum angular variation Δθ2(max) in the end region 62. It is sufficient that the conditions regarding the angular variation Δθ1 and the maximum angular variation Δθ2(max) described here are satisfied at least when the outer diameter of the electrode assembly 16 is at its maximum within the adjustable range of the outer diameter (here, the state shown in FIG. 4 or the like).

The larger the angular variation Δθ1 in the intermediate region 60, the easier it is to make the mesh shape formed by the intermediate region 60 denser in the axial direction. Therefore, it becomes easier to improve the shape retention of the electrode assembly 16 compared to the case where the angular variation Δθ1 is equal to or less than the maximum angular variation Δθ2(max). When the electrode assembly 16 has a mesh shape in the continuous conductive path 48, easily making the mesh shape formed by the intermediate region 60 denser allows the effect of expanding the circumferential range of the continuous conduction points with respect to the living tissue described above to be easily obtained.

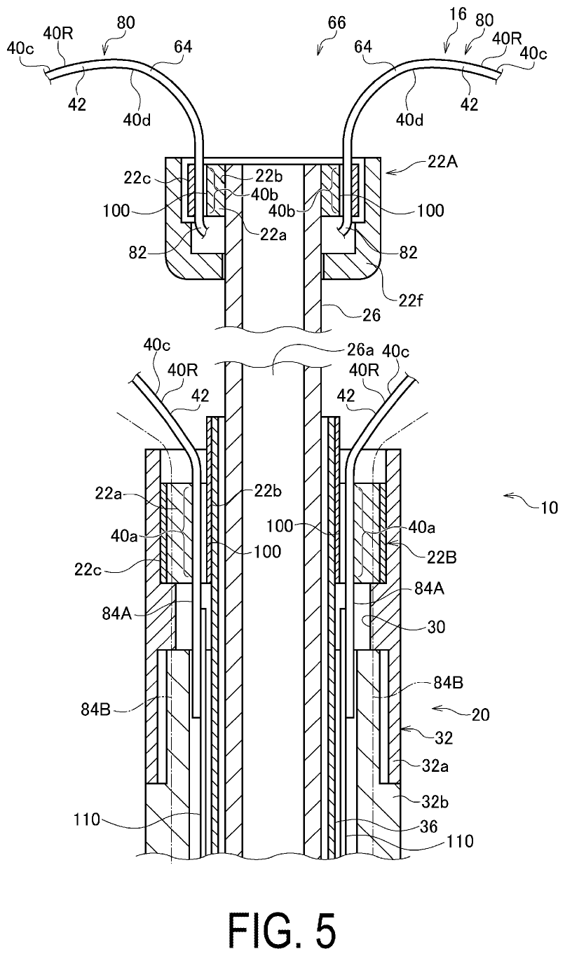

Description will be made with reference to FIGS. 4 and 5. Each of the plurality of spline portions 40L and 40R includes a folded portion 64 that is provided on the distal side from the distal-side bundling component 22A and is folded back in the axial direction. The folded portion 64 is provided on the above-mentioned distal-side extension portion 40d and extends radially outward from the distal end portion 40b side toward the proximal end portion 40a side in the longitudinal direction of the spline portion 40L or 40R and is fold back in the axial direction. The folded portion 64 is provided on the distal side from the distal end portions 40b of the spline portion 40L or 40R. This allows the plurality of spline portions 40L and 40R to form, in the electrode assembly 16, a recessed portion 66 that is recessed toward the proximal side in the radial inner side of the folded portion 64 of each of the plurality of spline portions 40L and 40R. The distal-side bundling component 22A is provided on the bottom side of the recessed portion 66 and forms the bottom of the recessed portion 66.

This provides a structure in which the distal-side bundling component 22A does not protrude further toward the distal side than each of the plurality of spline portions 40L and 40R. Therefore, compared to the case where the distal-side bundling component 22A protrudes toward the distal side from the plurality of spline portions 40L and 40R, strong contact of the distal-side bundling component 22A with the living tissue located on the distal side from the electrode assembly 16 can be avoided. At this time, each of the spline portions 40L and 40R of the electrode assembly 16, which can be flexibly deformed, can be brought into contact with the living tissue located on the distal side from the electrode assembly 16, and the contact with the living tissue becomes soft.

The wire forming the spline portion 40L or 40R is made of conductive metal, resin, or the like. The wire of the present embodiment is made of a shape memory alloy. Shape memory alloys are made of various alloys having shape memory properties, such as Ti—Ni alloys. The shape of the wire is memorized to form the spline portion 40L or 40R having the shape described above. This can easily obtain, even if the wires are deformed during the assembly process of the electrode assembly 16, the spline portions 40L and 40R having a desired shape by restoring the shape memorized by heating.

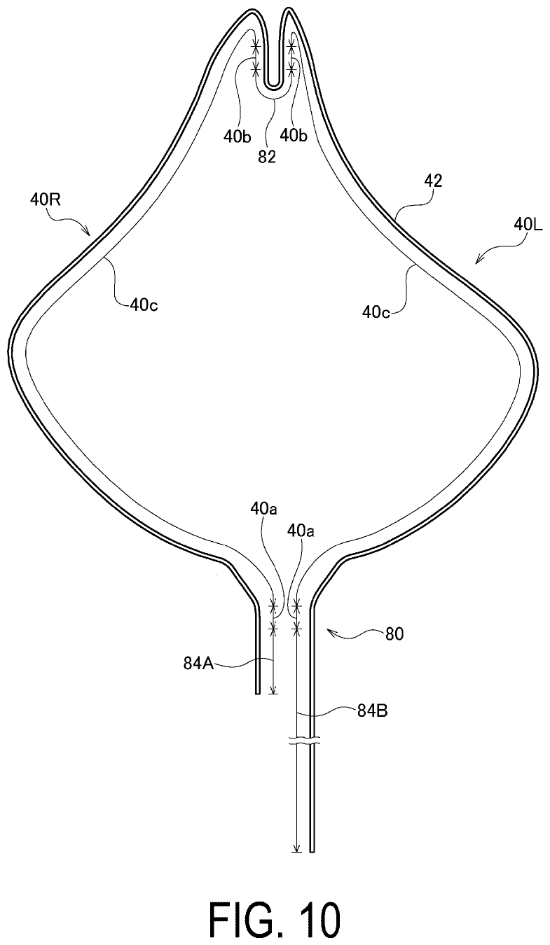

Next, the features related to (2) the end portions of the spline portion 40L and 40R mentioned above will be explained. Description will be made with reference to FIG. 10. The electrode catheter 10 includes at least one grouping linear member 80 that forms a set of spline portions composed of at least two spline portions 40L and 40R. The set of spline portions includes at least one clockwise spline portion 40R and one counterclockwise spline portion 40L. In order to satisfy this condition, in the present embodiment, one clockwise spline portion 40R and one counterclockwise spline portion 40L are included, but at least one of them may be included two or more. A set of spline portions is formed of separate parts of the grouping linear member 80. As described above, the grouping linear member 80 is made of a conductive wire. All of the spline portions 40L and 40R (thirty-two spline portions 40L and 40R in total in the present embodiment) include a plurality of sets (sixteen sets in the present embodiment) of spline portions. The individual set of spline portions is formed of individual grouping linear members 80.

The grouping linear member 80 includes, in addition to the plurality of spline portions 40L and 40R forming a set, a connecting portion 82 that connects the spline portions 40L adjacent in the longitudinal direction and a pair of terminal-side portions 84A and 84B extending outward in the longitudinal direction from the spline portions 40L and 40R that are outermost in the longitudinal direction.

The connecting portion 82 of the present embodiment connects the distal end portion 40b of the clockwise spline portion 40R and the distal end portion 40b of the counterclockwise spline portion 40L. The connecting portion 82 is formed folded back in the axial direction from the end portion 40b of one spline portion 40L connected by the connecting portion 82 toward the end portion 40b of the other spline portion 40R.

Description will be made with reference to FIGS. 5 and 10. Each of the pair of terminal-side portions 84A and 84B is pulled out toward the proximal side from an insertion hole 100 (described later) of the proximal-side bundling component 22B. One terminal-side portion 84A is conductively connected to a conductive wire 110 by soldering or the like. The conductive wire 110 is inserted into the main lumen 30 and electrically connects the external power supply device and the electrode portion 42 of the grouping linear member 80. Only the axis of the other terminal-side portion 84B is illustrated in FIG. 5. The other terminal-side portion 84B is inserted into a sub-lumen (not illustrated) formed in the shaft body 32 and opening into the main lumen 30.

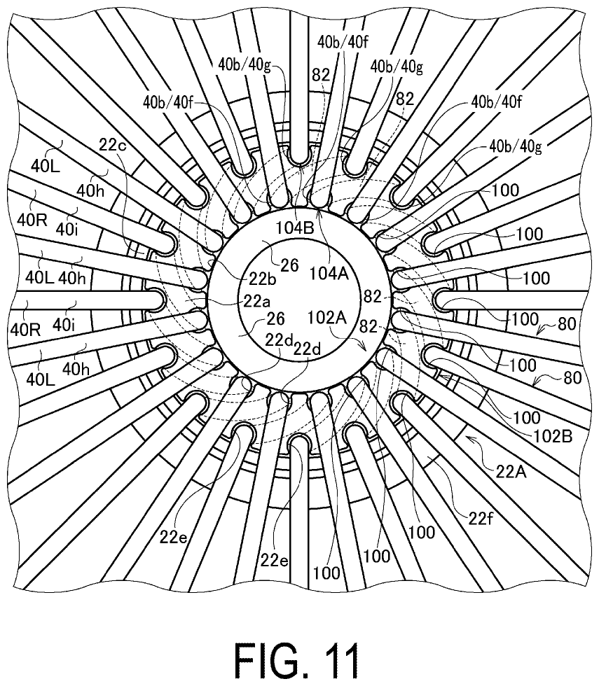

Description will be made with reference to FIGS. 5 and 11. The distal-side bundling component 22A includes a plurality of insertion holes 100 through which the distal end portions 40b of the plurality of spline portions 40L and 40R are inserted. The plurality of insertion holes 100 extends in the axial direction. In the present embodiment, the plurality of insertion holes 100 forms a plurality of rows of insertion hole groups 102A and 102B annularly arranged. The plurality of rows of insertion hole groups 102A and 102B includes an inner insertion hole group 102A and an outer insertion hole group 102B surrounding the inner insertion hole group 102A. The distal end portion 40b of the spline portion 40L or 40R may be inserted into the insertion hole 100 and then fixed to the distal-side bundling component 22A by adhesion, welding, interference fitting, or the like.

The distal-side bundling component 22A includes a ring member 22a, an inner cover member 22b disposed on the inner circumferential side of the ring member 22a, and an outer cover member 22c disposed on the outer circumferential side of the ring member 22a. Although the elongated member 26 also serves as the inner cover member 22b of the distal-side bundling component 22A, it may be separate from the elongated member 26. The ring member 22a includes a plurality of inner grooves 22d provided at intervals in the circumferential direction on the inner circumference of the ring member 22a and a plurality of outer grooves 22e provided at intervals in the circumferential direction on the outer circumference of the ring member 22a. The outer cover member 22c covers and closes the plurality of outer grooves 22e. The inner cover member 22b covers and closes the plurality of inner grooves 22d. The inner insertion hole group 102A is formed by the plurality of inner grooves 22d and the inner cover member 22b. The outer insertion hole group 102B is formed by the plurality of outer grooves 22e and the outer cover member 22c. The connecting portions 82 of the grouping linear member 80 are drawn out to the proximal side from each insertion hole 100 of the distal-side bundling component 22A. The distal-side bundling component 22A includes a protective member 22f that covers the connecting portion 82 of each of the plurality of grouping linear members 80.

The distal end portions 40b of the plurality of spline portions 40L and 40R form end portion groups 104A and 104B composed of a plurality of distal end portions 40b annularly arranged in the distal-side bundling component 22A. The end portion groups 104A and 104B are disposed in multiple rows in a nested shape when viewed along the axial direction. The term “nested shape” here refers to a state in which an inner ring-shaped end portion group is surrounded by an outer ring-shaped end portion group. The number of rows of end portion groups 104A and 104B in the present embodiment is two, but may be three or more.

The plurality of rows of end portion groups 104A and 104B includes an inner end portion group 104A and an outer end portion group 104B located on the outer side in the radial direction from the inner end portion group 104A. The outer end portion group 104B is arranged to surround the inner end portion group 104A. The inner end portion group 104A is composed of a plurality of inner end portions 40f, which is the distal end portions 40b of the plurality of spline portions 40L and 40R. The outer end portion group 104B is composed of a plurality of outer end portions 40g that are the distal end portions 40b of the plurality of spline portions 40L and 40R.

The outer end portions 40g and the distal end portions 40b of the plurality of spline portions 40L and 40R are annularly arranged around the axis C20 of the catheter shaft 20. The outer end portions 40g and the inner end portions 40f are arranged at positions shifted in the circumferential direction by an angular pitch P. The inner end portions 40f and the outer end portions 40g are alternately arranged at positions shifted in the circumferential direction by half the angular pitch P. The plurality of spline portions 40L and 40R forms a plurality of inner extension portions 40h extending radially outward in the radial direction from each inner end portion 40f and a plurality of outer extension portions 40i extending radially outward in the radial direction from each outer end portion 40g. Each of the plurality of inner extension portions 40h extends passing between the outer extension portions 40i adjacent in the circumferential direction when viewed along the axial direction. To satisfy this condition, the axes (not illustrated) of the plurality of inner extension portions 40h and the axes (not illustrated) of the plurality of outer extension portions 40i only need to extend without overlapping each other when viewed along the axial direction. In order to satisfy this condition, it is permissible for the inner extension portion 40h and the outer extension portion 40i to overlap at a location other than the mutual axes.

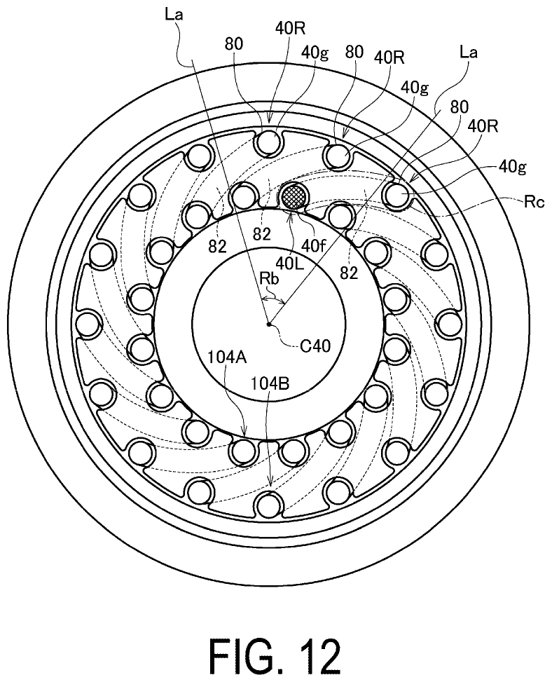

Description will be made with reference to FIG. 12. Although FIG. 12 is a cross-sectional view taken along line B-B of FIG. 4, hatching is omitted for convenience of explanation. The connecting portion 82 of the grouping linear member 80 connects the inner end portion 40f of one spline portion 40L and the outer end portion 40g of another spline portion 40R forming a pair with the one spline portion 40L. In a cross-section perpendicular to the axial direction passing through the inner end portion group 104A and the outer end portion group 104B, a circumferential range having two inner end portions 40f adjacent to each other on both sides in the circumferential direction with respect to the inner end portion 40f of the one spline portion 40L connected by the connecting portion 82 of the grouping linear member 80 is defined as a reference range Rb. The cross-section serving as a premise for determining this reference range Rb may be any cross-section orthogonal to the axial direction passing through the inner end portion group 104A and the outer end portion group 104B, and its axial position does not matter. This reference range Rb is a range defined by two circumscribed lines La that are centered on the axis C20 of the catheter shaft 20 and circumscribe each of the two inner end portions 40f when viewed along the axial direction. The reference range Rb is individually determined corresponding to each inner end portion 40f. Here, only one reference range Rb corresponding to one inner end portion 40f is illustrated, and the inner end portion 40f corresponding to the reference range Rb is hatched.

At this time, the outer end portion 40g (the outer end portion 40g within the range Rc) connected to the one inner end portion 40f by the connecting portion 82 is located outside the reference range Rb in the cross-section defining the reference range Rb corresponding to the inner end portion 40f. In the present embodiment, the entire outer end portion 40g is positioned outside the reference range Rb. In the present embodiment, at least part of the outer end portion 40g is positioned closest to the reference range Rb on one side in the circumferential direction outside the reference range Rb. The outer end portion 40g connected to one inner end portion 40f by the connecting portion 82 can also be said to be, of the plurality of outer end portions 40g, the end portion is 40g that is second closest to the one inner end portion 40f on one side in the circumferential direction. This condition may be satisfied by some of all the grouping linear members 80, preferably by two or more grouping linear members 80. In the present embodiment, this condition is satisfied by all the grouping linear members 80.

The effect of the electrode catheter 10 regarding the feature (2) will be described.

(A) A case in which only one row of annular end portion groups is formed by the end portions 40b of each of the plurality of spline portions 40L and 40R will be considered. In this case, the larger number of spline portions 40L and 40R forming the electrode assembly 16 makes it more difficult to have a space for disposing the end portions 40b adjacent in the circumferential direction in the end portion group. In this respect, the end portion groups 104A and 104B of the present embodiment are disposed in multiple rows in a nested shape. Therefore, compared to the case of arranging only one row of end portion groups, it is easier to have the space for disposing the end portions 40b forming the individual end portion groups 104A and 104B, making it easier to increase the number of spline portions 40L and 40R. It is advantageous in that the greater the number of spline portions 40L and 40R, the easier it is to widen the circumferential range of conduction points (contact portions of the electrode portions 42 provided on the spline portions 40L and 40R) with respect to living tissues. In particular, using the electrode catheter 10 for ablation is advantageous for annular cauterization.

(B) Each of the plurality of inner extension portions 40h extends passing between the outer extension portions 40i adjacent in the circumferential direction when viewed along the axial direction. This can easily avoid interference between the plurality of inner extension portions 40h and the plurality of outer extension portions 40i. As a result, the plurality of end portion groups 104A and 104B can be densely arranged while avoiding interference between the spline portions 40L and 40R, making it easier to increase the number of spline portions 40L and 40R.

The connecting portion 82 of the grouping linear member 80 connects the inner end portion 40f of one spline portion 40L and the outer end portion 40g of the other spline portion 40R. This can achieve, compared to the case of the adjacent distal end portions 40b connected by the connecting portion 82 in the same end portion group, a design to moderate the degree of bending at the connecting portion 82 without greatly increasing the circumferential interval between the adjacent distal end portions 40b. For example, this can be achieved by separating the outer end portion group 104B and the inner end portion group 104A apart in the radial direction to some extent, or connecting one inner end portion 40f and the outer end portion 40g outside the reference range Rb with respect to the one inner end portion 40f. By making the degree of bending at the connecting portion 82 moderate in this way, it is possible to reduce the difficulty of processing when forming the connecting portion 82 of the grouping linear member 80 by bending.

The outer end portion 40g, which is connected to the inner end portion 40f by the connecting portion 82, is positioned outside the reference range Rb. This can moderate the degree of bending at the connecting portion 82 compared to the case where the outer end portion 40g and the inner end portion 40f within the reference range Rb are connected.

A case where the connecting portion 82 of the grouping linear member 80 connects the outer end portion 40g and the inner end portion 40f outside the reference range Rb, which are far apart from the reference range Rb will be considered. In this case, it is necessary to increase the radial interval between the outer end portion group 104B and the inner end portion group 104A to have the space for disposing the connecting portion 82 of the grouping linear member 80. In this respect, the connecting portion 82 of the grouping linear member 80 of the present embodiment connects the outer end portion 40g and the inner end portion 40f that are closest to the reference range Rb outside the reference range Rb. Therefore, the inner end portion 40f of one spline portion 40L and the outer end portion 40g of the other spline portion 40R can be connected by the connecting portion 82 without greatly increasing the radial interval between the outer end portion group 104B and the inner end portion group 104A. As a result, the radial dimension of the distal-side bundling component 22A can be reduced.

A set of spline portions composed of a plurality of spline portions 40L and 40R is formed of one grouping linear member 80. Therefore, the number of parts required for the plurality of spline portions 40L and 40R can be reduced as compared with the case where the plurality of spline portions 40L and 40R forming a set are formed of individual linear members.

The distal end portions 40b of the two spline portions 40L and 40R formed of the grouping linear member 80 are inserted through different insertion holes 100 in the distal-side bundling component 22A. This condition is satisfied for each of the grouping linear members 80. This advantage will be explained.

An assembling operation of assembling the distal end portions 40b of the plurality of spline portions 40L and 40R to the distal-side bundling component 22A will be considered. When all the spline portions 40L and 40R are formed of individual linear members, if the distal end portions 40b of the spline portions 40L and 40R are simply inserted into the insertion holes 100 of the distal-side bundling component 22A, the spline portions 40L and 40R will rotate around the hole centers of the insertion holes 100. In this respect, according to the present embodiment, even if one spline portion 40L or 40R tries to rotate around the insertion hole 100, the distal end portion 40b of the other spline portion 40L or 40R formed of the same grouping linear member 80 comes into contact with another insertion hole 100, allowing the rotation to be restricted. Consequently, even if the distal end portions 40b of the spline portions 40L and 40R are not fixed to the distal-side bundling component 22A, it is possible to improve the position retention that the circumferential positions of the two spline portions 40L and 40R formed by the grouping linear member 80 are maintained at the target positions.

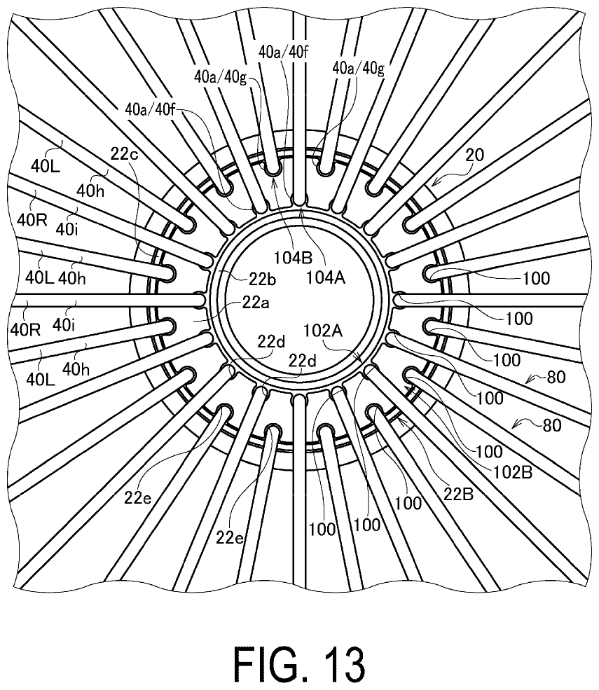

Description will be made with reference to FIGS. 5 and 13. So far, an example has been described in which the feature (2) is applied to the distal end portions 40b of the spline portions 40L and 40R and the distal-side bundling component 22A. The feature (2) in the present embodiment is commonly applied to the proximal end portions 40a of the spline portions 40L and 40R and the proximal-side bundling component 22B, with some exceptions. The features that are not commonly applied here refer to features related to the protective member 22f of the distal-side bundling component 22A and the connecting portion 82 of the grouping linear member 80.

The common features here refers to, for example, the features related to the end portion groups 104A and 104B and the extension portions 40h and 40i of the spline portions 40L and 40R in addition to the features related to the insertion hole 100, the ring member 22a, the cover members 22b and 22c of the distal-side bundling component 22A. For example, the proximal-side bundling component 22B also includes a plurality of insertion holes 100 through which the respective proximal end portions 40a of the plurality of spline portions 40L and 40R are inserted. The plurality of insertion holes 100 forms an inner insertion hole group 102A and an outer insertion hole group 102B annularly arranged. The proximal-side bundling component 22B also includes a ring member 22a and respective cover members 22b and 22c. The proximal end portions 40a of the plurality of spline portions 40L and 40R also form an inner end portion group 104A and an outer end portion group 104B each composed of a plurality of proximal end portions 40a. The plurality of spline portions 40L and 40R form an inner extension portion 40h extending from the inner end portions 40f that form the inner end portion group 104A and an outer extension portion 40i extending from the outer end portions 40g that form the outer end portion group 104B.

Only a brief description of such common features is given here, and details thereof are omitted. To understand the features commonly applied to the proximal end portion 40a of each spline portion 40L or 40R, and the like, the terms “distal end portion 40b” and “distal-side bundling component 22A” in the description of the features applied to the distal end portion 40b, and the like of each spline portion 40L or 40R described above may be replaced with the terms “proximal end portion 40a” and “proximal-side bundling component 22B”. For example, since the proximal end portions 40a of the plurality of spline portions 40L and 40R form the nested end portion groups 104A and 104B, it is easy to increase the number of the spline portions 40L and 40R as in (A) above. Each of the plurality of inner extension portions 40h extending from the proximal end portion 40a of each of the spline portions 40L and 40R extends passing between the plurality of outer extension portions 40i extending from the proximal end portion 40a. Therefore, as in (B) above, it becomes easy to increase the number of spline portions 40L and 40R.

Such features common to the bundling components 22A and 22B are applied to at least one of the distal-side bundling component 22A and the proximal-side bundling component 22B. The features related to the end portions 40a and 40b of the spline portions 40L and 40R may also be applied to at least one of the proximal end portion 40a and the distal end portion 40b.

Next, modifications of each component described so far will be described.

The electrode catheter 10 may be used for various types of ablation (PFA, high-frequency ablation, or the like) for annularly cauterizing living tissues or may be used for various other treatments and various examinations (electrocardiography and the like). The treatment target of the living body by the electrode catheter 10 is not limited to circulatory organs such as the heart and pulmonary veins, and may be various organs such as digestive organs.

The number of spline portions 40L and 40R is not particularly limited, and may be at least two. In this case, the number of counterclockwise spline portions 40L and the number of clockwise spline portions 40R may be one (two in total). In addition, all the spline portions may include only one of the counterclockwise spline portion 40L and the clockwise spline portion 40R and may also include a spline portion extending linearly in the axial direction from the distal end portion 40b side toward the proximal end portion 40a side.

The spline portions 40L and 40R may be formed of a linear member including a wire (which may or may not be conductive) and a coating layer that covers the wire. In this case, the electrode portions 42 may be provided in the spline portions 40L and 40R by making the coating layer itself conductive. In addition, a plurality of electrode portions 42 of the spline portions 40L and 40R may be provided as ring electrodes or the like attached to the spline portions 40L and 40R at intervals in the longitudinal direction of the spline portion 40L and 40R. When the wire of the linear member is made of a shape memory alloy such as Ni—Ti, it may be plated with gold, platinum, or the like to prevent electrolysis and hydrogen embrittlement due to energization.

The number of spline portions (the number of spline portions forming a set) of the grouping linear member 80 is not limited to two, and may be three or more. The number of grouping linear members 80 is not particularly limited, and may be either singular or plural. The spline portion of the grouping linear member 80 may be composed only of a plurality of the clockwise spline portions 40R or may be composed only of a plurality of the counterclockwise spline portions 40L. One or more spline portions 40L and 40R out of all the spline portions 40L and 40R may be formed of linear members corresponding to the spline portions 40L and 40R in a one-to-one correspondence instead of the grouping linear member 80.

The continuous conductive path 48 does not have to be continuous along the entire circumference of the axis C20 of the catheter shaft 20. The electrode assembly 16 may include only a single row of zigzag patterns 46 without the mesh shape of continuous conductive paths 48. The magnitude relationship between the angular variation Δθ1 in the intermediate region 60 and the angular variation Δθ2 in the end region 62 of the plurality of spline portions 40L and 40R is not particularly limited. The angular variation Δθ1 may be equal to or less than the maximum angular variation Δθ2(max) in the end region 62.

In the embodiment, it can be said that an example has been described in which one end portion of the distal end portion 40b and the proximal end portion 40a of each of the plurality of spline portions 40L and 40R forms a first end portion group composed of one set of a plurality of end portions, and the other end portions of them forms a second end portion group composed of a set of plurality of other end portions. In the embodiment, an example has been described in which both the first end portion group and the second end portion group are disposed in multiple rows in a nested manner. Alternatively, only one of the first end portion group and the second end portion group may be disposed in multiple rows in a nested manner, and the other of them may be arranged in only one row. In the plurality of spline portions, the plurality of inner extension portions 40h and the plurality of outer extension portions 40i may extend passing through positions where they overlap each other when viewed along the axial direction.

Described is an example in which the grouping linear member 80 includes the connecting portion 82 that connects the distal end portion 40b of one spline portion (counterclockwise spline portion 40L) and the distal end portion 40b of another spline portion (clockwise spline portion 40R). In addition, the grouping linear member 80 may include the connecting portion 82 that connects the proximal end portion 40a of one spline portion and the proximal end portion 40a of another spline portion. The grouping linear member 80 may include one or both of the connecting portion 82 connecting the distal end portions 40b of the different spline portions and the connecting portion 82 connecting the proximal end portions of the different spline portions.

The combination of the end portions of different spline portions connected by the connecting portion 82 is not particularly limited. For example, the connecting portion 82 may connect the end portions of adjacent spline portions forming an annular end portion group in the same row. The connecting portion 82 may connect the outer end portion 40g of another of the spline portions within the reference range Rb corresponding to the inner end portion 40f of one spline portion and the inner end portion 40f. The connecting portion 82 may connect the inner end portion 40f with the outer end portion 40g other than the nearest outer end portion 40g outside the reference range Rb.

Specific examples of each bundling component 20A and 20B are not particularly limited. The end portions of the spline portions 40L and 40R do not need to be inserted through the insertion holes 100 of the bundling components 20A and 20B and may be fixed to the bundling components 20A and 20B by adhesion or the like on the outside thereof.

The embodiments and modifications described above are examples. Technical ideas obtained by abstraction thereof should not be interpreted as limited to the contents of the embodiments and modifications. Numerous design changes, such as modification, addition, and deletion of constituent elements, can be made to the contents of the embodiments and modifications. In the embodiments described above, the content in which such design changes can be made has been emphasized with expressions such as “of the embodiment.” However, design changes are also possible even in the content without such an expression. Hatching in sections of the drawings does not limit the material of a hatched object. Structures and numerical values referred to in the embodiments and the modifications naturally include those that can be regarded as the same in consideration of manufacturing errors and the like.

When generalizing the technical idea embodied by the embodiments and modifications, it can be said that the technical idea described in the following second aspect is included in addition to the first aspect described above.

An electrode catheter according to the second aspect includes: a catheter shaft; and an electrode assembly and a distal-side bundling component disposed at least partially distal to the catheter shaft, the electrode assembly including a plurality of spline portions of which the proximal end portions are bundled by the catheter shaft and the distal end portions are bundled by the distal-side bundling component, one end portion of the distal end portion and the proximal end portion of each of the plurality of spline portions forming an end portion group composed of a plurality of one end portions annularly arranged, and the end portion group being disposed in multiple rows in a nested manner when viewed along the axial direction.

In realizing the electrode catheter of the first aspect, the end portion groups may not be disposed in multiple rows in a nested manner unlike the electrode catheter of the second aspect. In realizing the electrode catheter of the second aspect, the plurality of spline portions may not include a clockwise spline portion and a counterclockwise spline portion unlike the electrode catheter of the first aspect.

Any combination of the components is also effective. For example, an embodiment may be combined with any description of another embodiment, or a modified form may be combined with any description of the embodiment and other modifications.

While preferred embodiments of the disclosure have been described above, it is to be understood that variations and modifications will be apparent to those skilled in the art without departing from the scope and spirit of the disclosure. The scope of the disclosure, therefore, is to be determined solely by the following claims.

1. An electrode catheter, comprising:
a catheter shaft; and
an electrode assembly and a distal-side bundling component disposed at least partially distal to the catheter shaft, wherein
the electrode assembly comprises a plurality of spline portions each having a proximal end portion bundled by the catheter shaft and a distal end portion bundled by the distal-side bundling component, and
the plurality of spline portions comprises, when viewed from a distal side in an axial direction of the catheter shaft, a counterclockwise spline portion extending counterclockwise from a side of the distal end portion toward a side of the proximal end portion and a clockwise spline portion extending clockwise from the side of the distal end portion toward the side of the proximal end portion.

2. The electrode catheter according to claim 1, wherein the clockwise spline portion and the counterclockwise spline portion are provided intersecting in a contactable manner.

3. The electrode catheter according to claim 1, wherein the electrode assembly has a mesh shape formed by a plurality of the clockwise spline portions and a plurality of the counterclockwise spline portions.

4. The electrode catheter according to claim 1, wherein
each of the plurality of spline portions is provided with an electrode portion, and
the electrode assembly can form a continuous conductive path that is continuous with the electrode portion of the clockwise spline portion and the electrode portion of the counterclockwise spline portion.

5. The electrode catheter of claim 4, wherein the continuous conductive path is continuous along an entire circumference around a centerline of the catheter shaft.

6. The electrode catheter of claim 4, wherein the electrode assembly has a mesh shape formed by a plurality of the clockwise spline portions and a plurality of the counterclockwise spline portions in the continuous conductive path.

7. The electrode catheter according to claim 3, wherein
the electrode assembly comprises an intermediate region forming the mesh shape and a pair of end regions provided on respective both sides of the intermediate region in the axial direction, and
an angular variation Δθ1 per unit axial length in at least part of the intermediate region is larger than a maximum angular variation Δθ2(max) per unit axial length in the end region.

8. The electrode catheter according to claim 1, wherein
one end portion of the distal end portion and the proximal end portion of each of the plurality of spline portions forms an end portion group composed of a plurality of the one end portions annularly arranged, and
a plurality of rows of the end portion groups is disposed in a nested manner when viewed along the axial direction.

9. An electrode catheter comprising:
a catheter shaft; and
an electrode assembly and a distal-side bundling component disposed at least partially distal to the catheter shaft, wherein
the electrode assembly comprises a plurality of spline portions each having a proximal end portion bundled by the catheter shaft and a distal end portion bundled by the distal-side bundling component,
one end portion of the distal end portion and the proximal end portion of each of the plurality of spline portions forms an end portion group composed of a plurality of the one end portions annularly arranged, and
a plurality of rows of the end portion groups is disposed in a nested manner when viewed along the axial direction.

10. The electrode catheter according to claim 9, wherein
the plurality of rows of the end portion groups comprises an inner end portion group and an outer end portion group,
the plurality of spline portions forms a plurality of inner extension portions extending radially outward in a radial direction from the inner end portion group and a plurality of outer extension portions extending radially outward in the radial direction from the outer end portion group, and
each of the plurality of inner extension portions extends passing between the outer extension portions adjacent to each other in a circumferential direction when viewed along the axial direction.

11. The electrode catheter according to claim 9, wherein
the plurality of rows of the end portion groups comprises an inner end portion group composed of a plurality of inner end portions that are the plurality of the one end portions and an outer end portion group composed of a plurality of outer end portions that are the plurality of the one end portions,
the electrode catheter comprises at least one grouping linear member forming a set of spline portions composed of at least two of the spline portions, and
the grouping linear member comprises a connecting portion connecting an inner end portion of the inner end portions of one of the spline portions and an outer end portion of the outer end portions of another of the spline portions.

12. The electrode catheter according to claim 11, wherein
in a cross-section perpendicular to the axial direction passing through the inner end portion group and the outer end portion group, a circumferential range having two of the inner end portions adjacent to each other on both sides in a circumferential direction with respect to the inner end portions of the one of the spline portions is defined as a reference range, and
at least some of the outer end portions connected to the inner end portion of the one of the spline portions by the connecting portion is located outside the reference range.

13. The electrode catheter according to claim 12, wherein at least some of the outer end portions connected to the inner end portion of the one of the spline portions by the connecting portion is located closest to the reference range on one side in the circumferential direction outside the reference range.

14. The electrode catheter according to claim 1, comprising at least one grouping linear member forming a set of spline portions composed of at least two of the spline portions.

15. The electrode catheter according to claim 14, wherein the set of spline portions comprises at least the clockwise spline portion and the counterclockwise spline portion.

16. The electrode catheter according to claim 14, wherein
the distal-side bundling component comprises a plurality of insertion holes, the distal end portion of each of the plurality of spline portions being inserted through an insertion hole of the plurality of insertion holes, and
the distal end portion of each of the two of the spline portions formed by the grouping linear member is inserted through the insertion hole different in the distal-side bundling component.

17. The electrode catheter according to claim 1, wherein each of the plurality of spline portions comprises a folded portion that is provided on the distal side from the distal-side bundling component and is folded back in the axial direction.
